(19) United States
(12) Patent Application Publication
(10) Pub. No.: US 2014/0188171 A1
(43) Pub. Date: Jul. 3, 2014

(54) BONE FIXING DEVICE

(71) Applicant: Meng Feng Huang, Shengang Township (TW)

(72) Inventor: Meng Feng Huang, Shengang Township (TW)

(21) Appl. No.: 13/727,590

(22) Filed: Dec. 27, 2012

(51) Int. Cl. A61B 17/70

(52) U.S. Cl. CPC A61B 17/7035
USPC 606/278

(57) ABSTRACT

A bone fixing device includes a tubular member that has an end having an inside surface defining a constraint section. A fixing member has opposite ends respectively forming a movable joint section that is receivingly engageable with the constraint section and a threaded section that is attachable to a bone or a retention section that is snap-fit over a bone. The movable joint section forms a constraint face corresponding to the constraint section. The constraint section and the constraint face collectively constrain a swing direction of the fixing member. The movable joint section has a top end with which a constraint member is abuttingly engageable. Further, the tubular member forms an elongate constraint slot in a portion adjacent to the constraint section for constraining swing angle of the fixing member. The constraint slot has a length that is proportional to the swing angle of the fixing member.

### TECHNICAL FIELD OF THE INVENTION

The present invention generally relates to a bone fixing device, and more particularly to a stable and comfortable bone fixing device.

### DESCRIPTION OF THE PRIOR ART

Bone fixing devices are widely used in medical treatments. For example, a bone fixing device is applicable to fixing vertebra bones. As shown in FIG. 1, which is a schematic view showing the prior art device, the prior art bone fixing device has a movable joint section 91, which comprises a universal joint. In a practical application, the non-fixed direction of the joint may cause undesired shifting of a tubular member 92 and a fixing member 93 in opposite directions, whereby locking may occur after the device is mounted, making it impossible to move. Thus, such a device, when put into use, may not meet the need of a patient for stretching a bone in a given direction after the bone is fixed so that the bone joint of the patient that takes the surgery operation become immobile, which leads to stiffening and discomfort of the patient.

Thus, it is desired to provide a solution to overcome such a problem, which is a goal that improvement needs to be made in the industry.

### SUMMARY OF THE INVENTION

The present invention aims to provide a bone fixing device that is stable and comfortable to a patent in order to overcome the above discussed problems.

The primary object of the present invention is to provide a solution that overcomes the problem that the conventional bone fixing device that comprises a movable joint section that is a universal joint, which in an actual application, provides a non-fixed direction of joint that causes undesired shifting of a tubular member and a fixing member in opposite direction, but may get locked and thus immobile when installed so as not to meet the need of mobility and stretching of the bone after fixing and the patient taking the surgery feeling stiffening and uncomfortable and that also shows stability and comfortableness in use.

To achieve the object, the present invention provides a bone fixing device, which comprises a tubular member, a fixing member, and a constraint member.

The tubular member has an end having an inside surface defining a constraint section. The fixing member has opposite ends respectively forming a movable joint section that is receivingly engageable with the constraint section and a threaded section that is attachable to a bone or a retention section that is snap-fit over a bone. The movable joint section forms a constraint face corresponding to the constraint section. The constraint section and the constraint face collectively constrain a swing direction of the fixing member. The movable joint section has a top end with which the constraint member is abuttingly engageable to retain the fixing member in the tubular member. Further, the tubular member forms an elongate constraint slot in a portion adjacent to the constraint section for constraining swing angle of the fixing member. The constraint slot has a length that is proportional to the swing angle of the fixing member. The tubular member forms a coupling section to which the constraint member is locked or fixed, wherein the constraint member is a locking element or a bar.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective view showing the prior art.

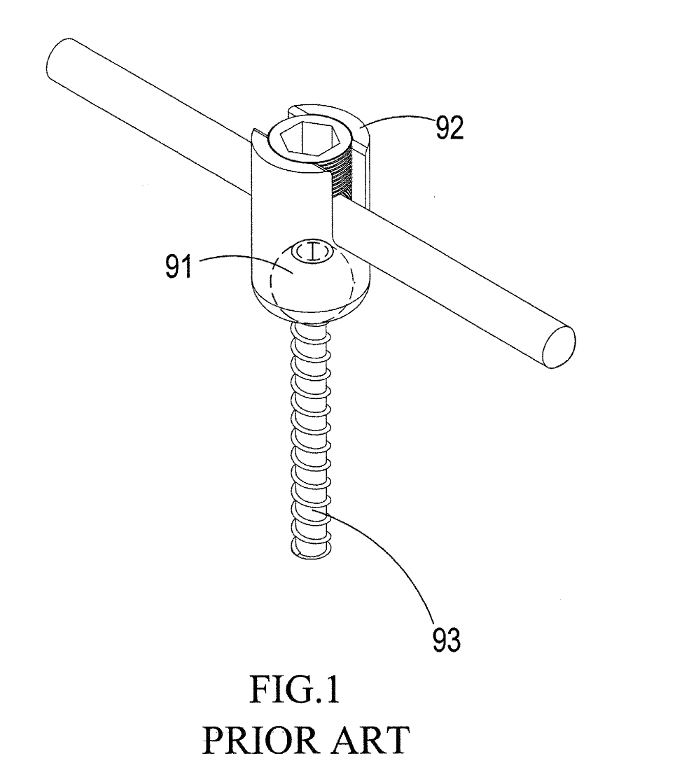

FIG. 2 is an exploded view of a preferred embodiment of the present invention.

FIG. 3 is a cross-sectional view of the preferred embodiment of the present invention.

FIG. 4A is a view illustrating a swinging operation of the preferred embodiment of the present invention.

FIG. 4B is a partial enlarged view illustrating the swinging operation of the preferred embodiment of the present invention.

FIG. 4C is another view illustrating the swinging operation of the preferred embodiment of the present invention.

FIG. 4D is another partial enlarged view illustrating the swinging operation of the preferred embodiment of the present invention.

FIG. 4E is a further view illustrating the swinging operation of the preferred embodiment of the present invention.

FIG. 4F is a further partial enlarged view illustrating the swinging operation of the preferred embodiment of the present invention.

FIG. 5A is a view illustrating an attached condition of the preferred embodiment of the present invention.

FIG. 5B is another view illustrating the attached condition of the preferred embodiment of the present invention.

FIG. 6 is a cross-sectional view of a second preferred embodiment of the present invention.

FIG. 7 is a cross-sectional view of a third preferred embodiment of the present invention.

FIG. 8 is a view showing a fourth embodiment of the present invention.

FIG. 9 is a view showing a fifth embodiment of the present invention.

FIG. 10 is a view illustrating snap-fitting of a sixth embodiment of the present invention.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

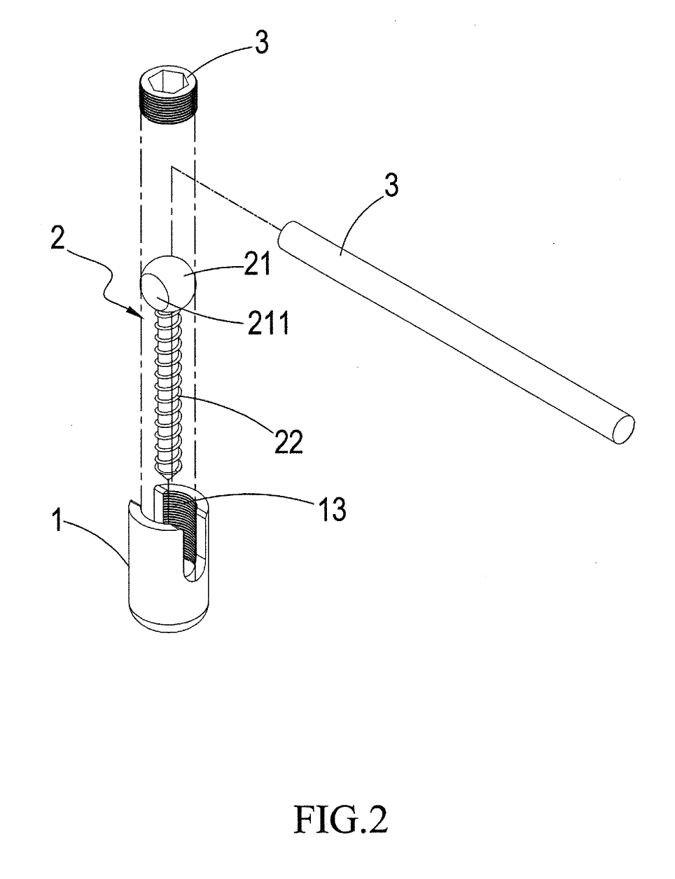

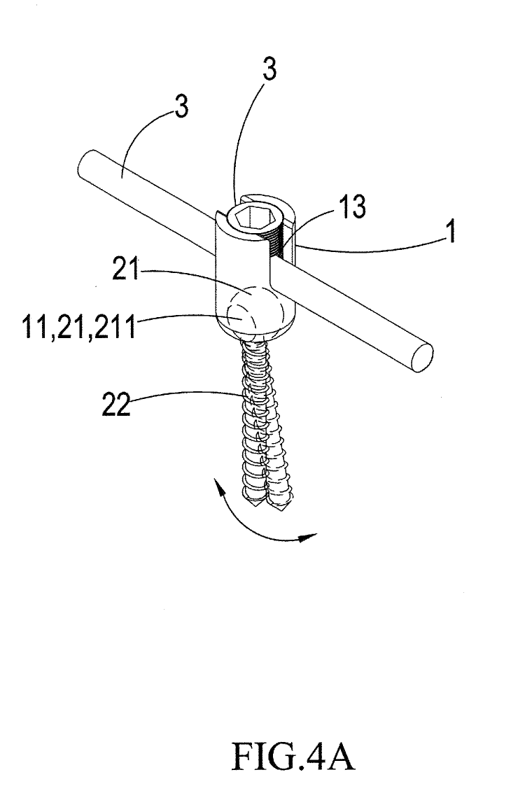

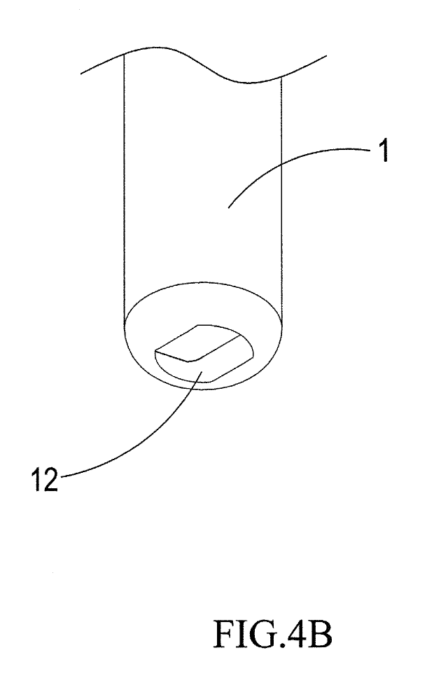

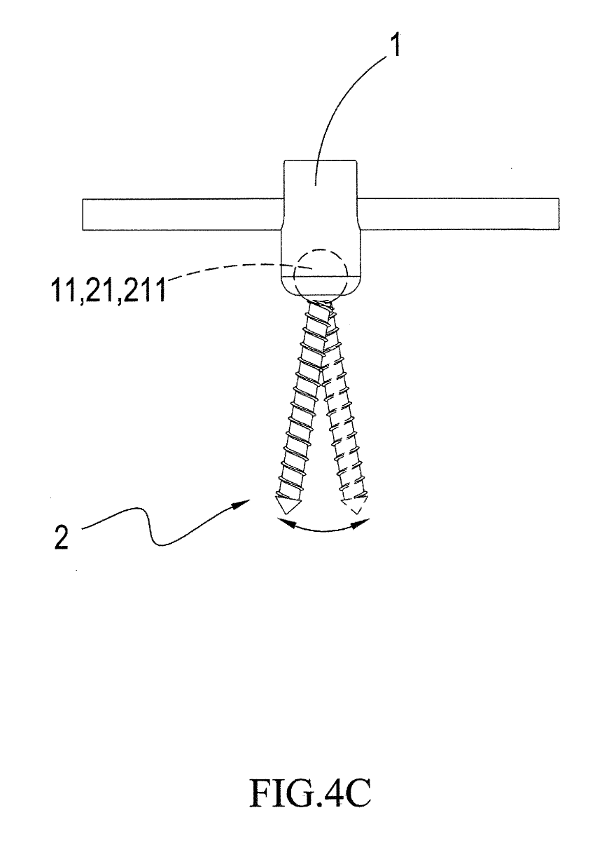

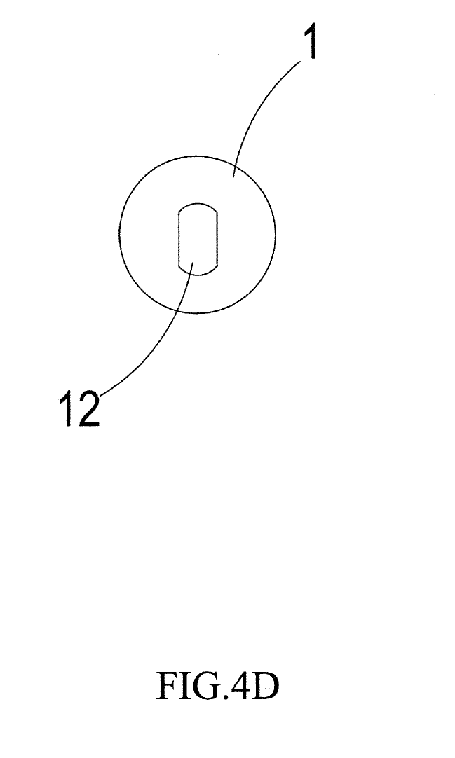

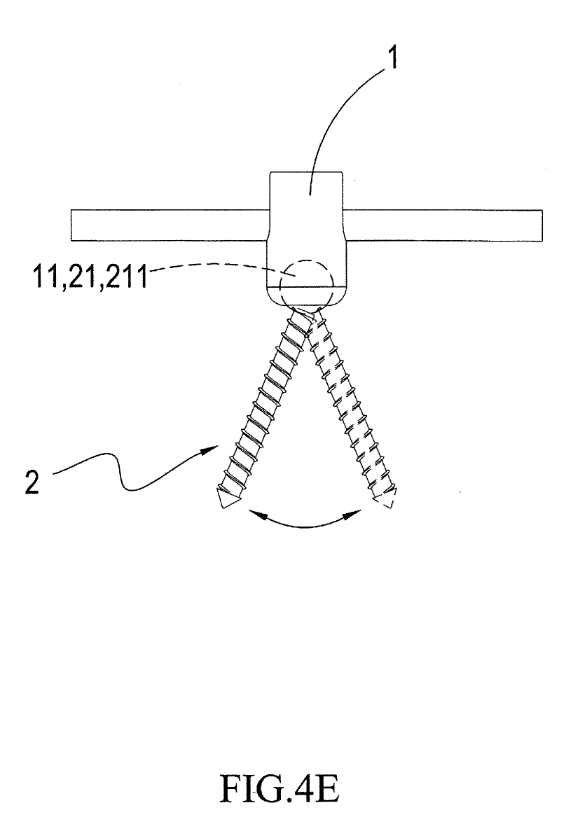

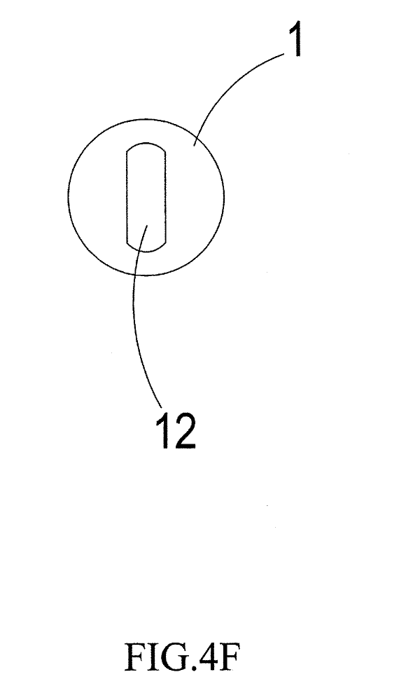

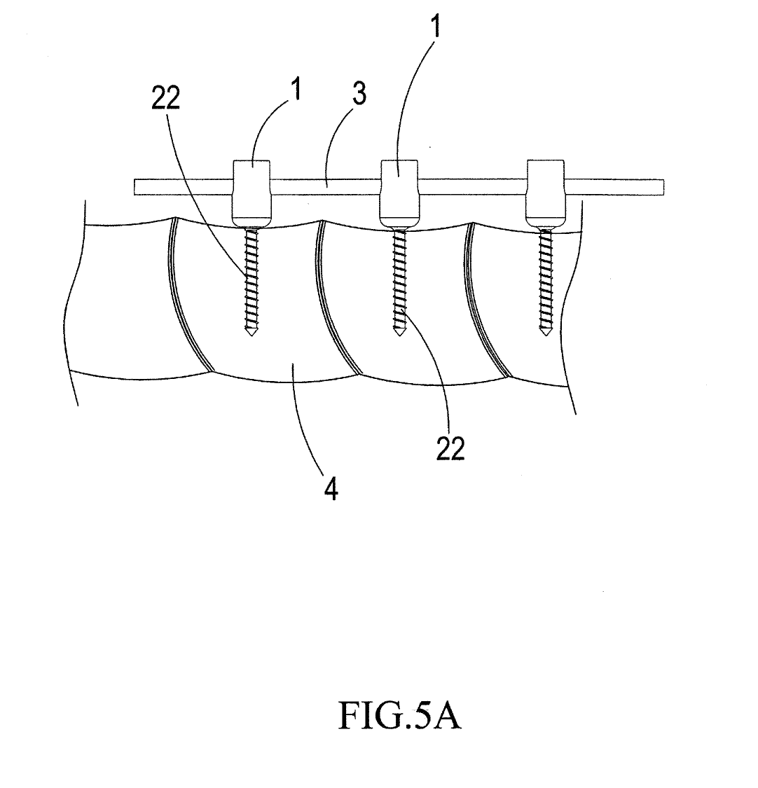

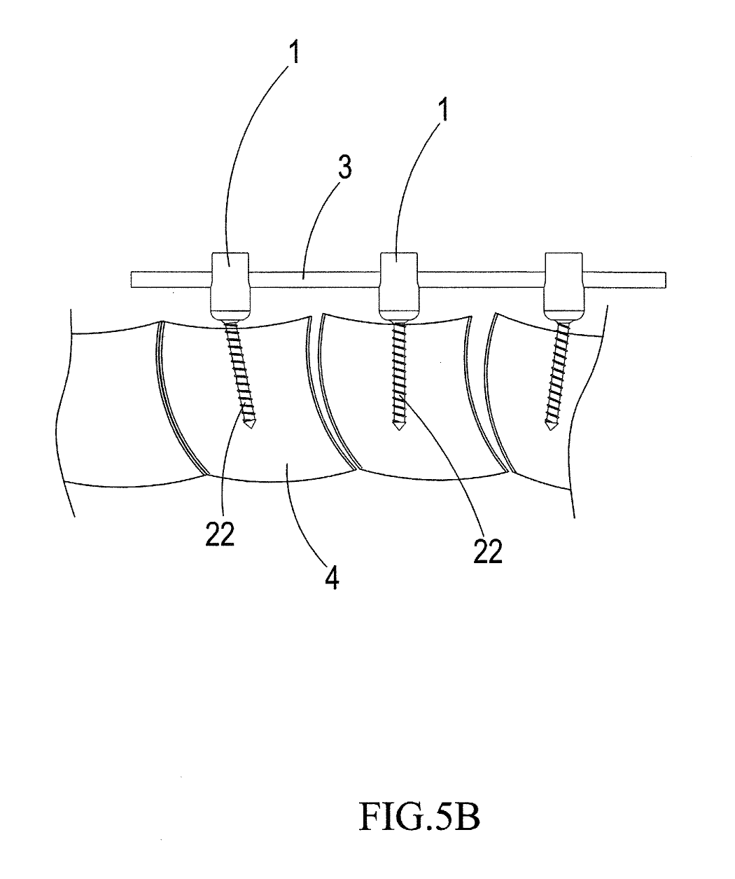

Referring to FIGS. 2, 3, 4A, 5A, and 5B, which are respectively an exploded view of a preferred embodiment of the present invention, a cross-sectional view of the preferred embodiment of the present invention, a view illustrating a swinging operation of the preferred embodiment of the present invention, a view illustrating an attached condition of the preferred embodiment of the present invention, and another view illustrating the attached condition of the preferred embodiment of the present invention, these drawings clearly show a bone fixing device according to the present invention, which comprises at least a tubular member 1, at least one fixing member 2, and at least one constraint member 3. The tubular member 1 has an end having an inside surface defining at least one constraint section 11. The fixing member 2 has opposite ends respectively forming a movable joint section 21 that is receivingly engageable with the constraint section 11 and a threaded section 22 that is attachable to a bone 4. The movable joint section 21 forms at least one constraint face 211 corresponding to the constraint section 11. The constraint section 11 and the constraint face 211 collectively define and constrain a swing direction of the fixing member 2. The movable joint section 21 has a top end with which the constraint member 3 is abuttingly engageable. Further, the tubular member 1 forms an elongate constraint slot 12 in a portion adjacent to the constraint section 11 for constraining swing angle of the fixing member 2. The constraint slot 12 has a length that is proportional to or corresponds to the swing angle of the fixing member 2.

The tubular member 1 also forms at least one coupling section 13 to which the constraint member 3 is fixed, wherein the constraint member 3 is a locking element.

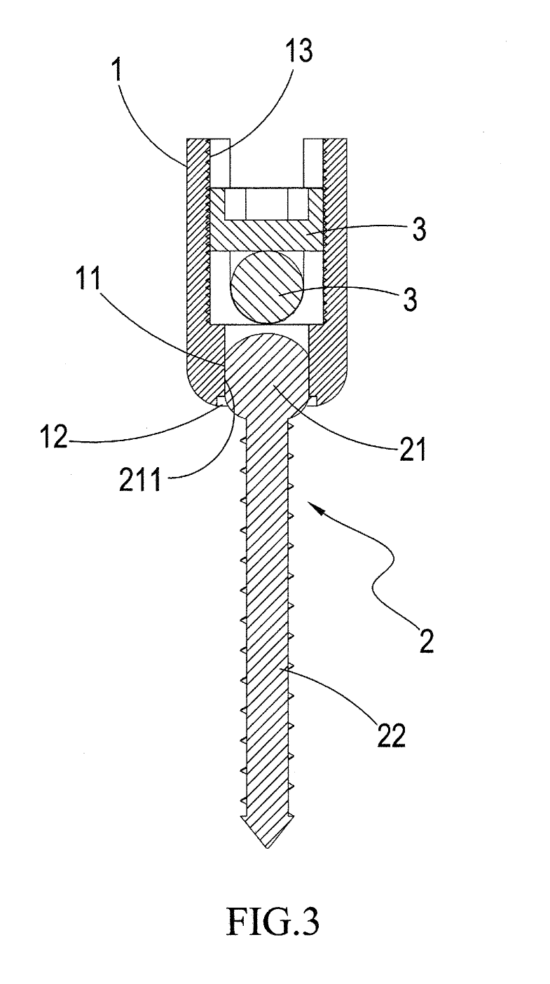

With the above described structure and arrangement, the operation of the present invention will be described as follows, with reference to FIGS. 3, 4A, 4B, 4C, 4D, 4E, 5A, and 5B, which are respectively a cross-sectional view of the preferred embodiment of the present invention, a view illustrating a swinging operation of the preferred embodiment of the present invention, a partial enlarged view illustrating the swinging operation of the preferred embodiment of the present invention, another view illustrating the swinging operation of the preferred embodiment of the present invention, another partial enlarged view illustrating the swinging operation of the preferred embodiment of the present invention, a further view illustrating the swinging operation of the preferred embodiment of the present invention, a view illustrating an attached condition of the preferred embodiment of the present invention, and another view illustrating the attached condition of the preferred embodiment of the present invention, these drawings clearly show the constraint section 11 and the constraint slot 12 collectively constrain the fixing member 2 to swing in a given horizontal direction and thus a benefit of having the movement confined in a predetermined range can be achieved. Further, the constraint member 3 comprises a bar and a locking element operable in combination with each other. The coupling section 13 formed in the tubular member 1 receives the bar and the locking element to be coupled and fixed thereto. The instant embodiment arranges a bar like constraint member 3 to abut the movable joint section 21 and uses a locking element like constraint member 3 mounted to the coupling section 13 to prevent the bar like constraint member 3 from detaching. Further, the length of the constraint slot 12 has a range that is proportional to a desired angular range of swing of the fixing member 2 so as to control the range of movement that the bone 4 is allowed, making the use comfortable and preventing situations of stiffening and sideway shifting.

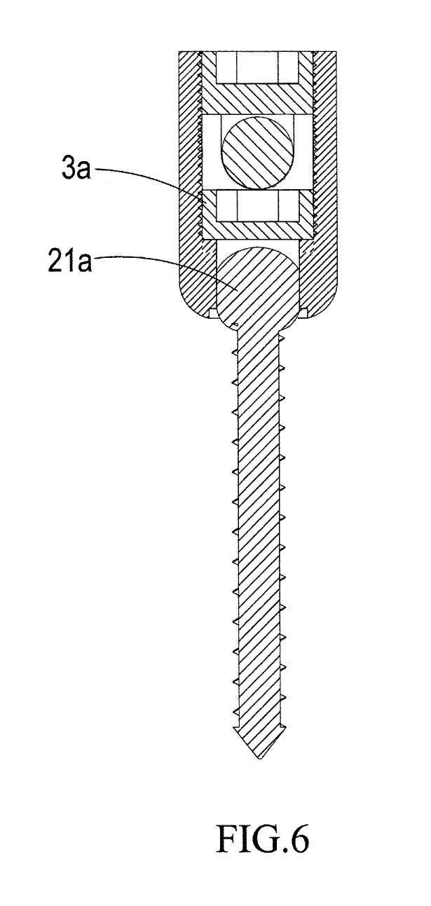

Also referring to FIG. 6, which is a cross-sectional view of a second preferred embodiment of the present invention, the drawing clearly shows that a movable joint section 21 a is provided with an additional lockable constraint member 3a, above which the bar of a bar like constraint member 3a and a locking element like constraint member 3a are mounted to improve the locking performance thereof.

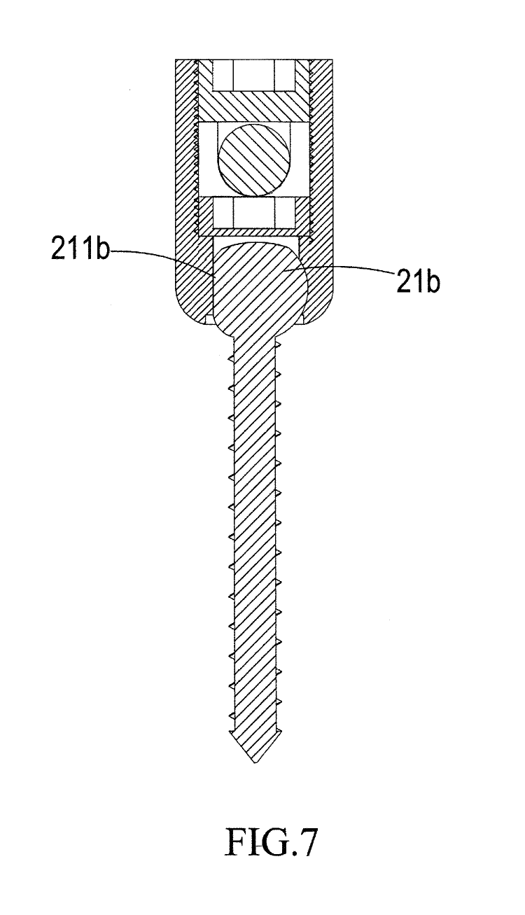

Also referring to FIG. 7, which is a cross-sectional view of a third preferred embodiment of the present invention, the drawing clearly shows a movable joint section 21b has one side forming a constraint face 211b to provide a desired effect of constraining.

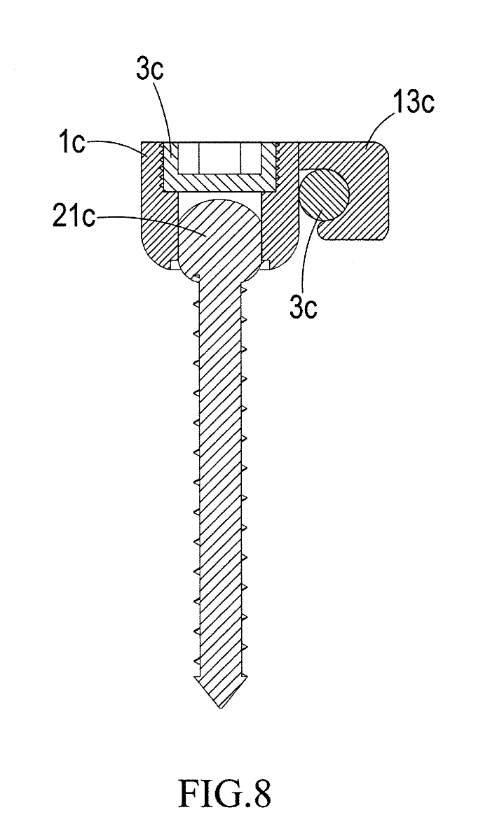

Also referring to FIG. 8, which is a view showing a fourth embodiment of the present invention, the drawing clearly shows that a constraint member 3c that is arranged above a movable joint section 21c is a locking element for securely fixing the movable joint section 21c. Further, a tubular member 1c forms a coupling section 13c projecting therefrom to allow a bar like constraint member 3c to extend therethrough for achieving an effectiveness of connecting one tubular member 1c to another tubular member 1c in a cascade form.

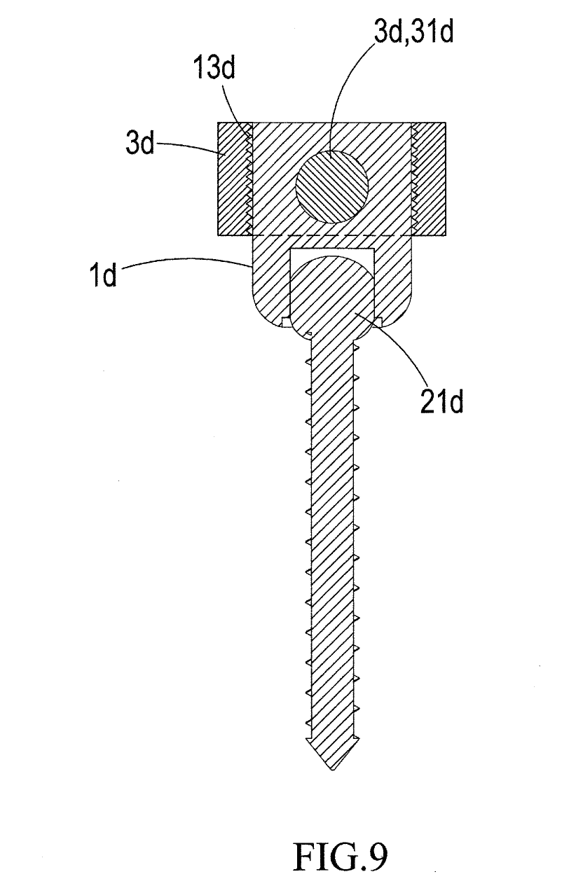

Referring to FIG. 9, which is a view showing a fifth embodiment of the present invention, the drawing clearly shows a tubular member 1d has an outside tubular wall that forms a coupling section 13d to which a constraint member 3d is coupled and fixed above the movable joint section 21d. The constraint member 3d that is in the form of a locking element forms a through hole 31 d that receives a bar like constraint member 3d to extend therethrough for the purposes of both fixing and saving material.

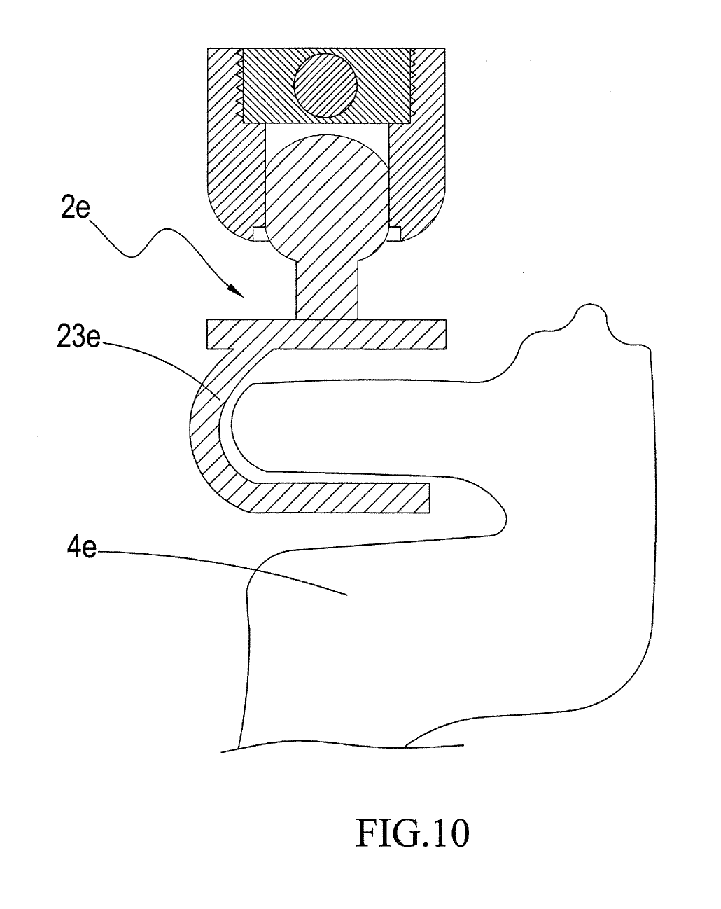

Referring to FIG. 10, which is a view illustrating snap-fitting of a sixth embodiment of the present invention, the drawing clearly shows that a fixing member 2e has a distal end forming a retention section 23e to be snap-fit over a bone 4e so that attaching to the bone 4e can be achieved with the retention section 23e.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A bone fixing device, comprising:
at least a tubular member, which has an end having an inside surface defining at least one constraint section;
at least one fixing member, which has an end forming a movable joint section that is receivingly engageable with the constraint section, the movable joint section forming at least one constraint face corresponding to the constraint section; and
at least one constraint member, which is abuttingly engageable with the movable joint section.

2. The bone fixing device according to claim 1, wherein the fixing member has an opposite end forming a threaded section that is attachable to a bone.

3. The bone fixing device according to claim 1, wherein the fixing member has a distal end forming retention section that is snap-fit over a bone.

4. The bone fixing device according to claim 1, wherein the constraint section and the constraint face collectively constrain a swing direction of the fixing member.

5. The bone fixing device according to claim 1, wherein the tubular member forms an elongate constraint slot in a portion adjacent to the constraint section for constraining swing angle of the fixing member, the constraint slot having a length that is proportional to the swing angle of the fixing member.

6. The bone fixing device according to claim 1, wherein the tubular member forms at least one coupling section to which the constraint member is fixed.

7. The bone fixing device according to claim 6, wherein the coupling section receives the constraint member to be locked thereon.

8. The bone fixing device according to claim 7, wherein the constraint member is a locking element.

9. The bone fixing device according to claim 6, wherein the coupling section receives and retains the constraint member therein

10. The bone fixing device according to claim 9, wherein the constraint member comprises a bar.
